(19) United States
(12) Patent Application Publication
(10) Pub. No.: US 2026/0325606 A1
(43) Pub. Date: Oct. 22, 2026

(54) NOVEL COMBINATIONS OF ACTIVE INGREDIENTS, COMPOSITIONS CONTAINING THEM AND THEIR USE IN THERAPY

(71) Applicant: GLYCORES 2000 SRL, Milano (MI) (IT)

(72) Inventors: Pasqua ORESTE, Milano (Mi) (IT); Giorgio Zoppetti, Milano (IT); Berardino Vaira, Bologna (BO) (IT)

(73) Assignee: GLYCORES 2000 SRL, Milano (MI) (IT)

(21) Appl. No.: 19/486,765

(22) PCT Filed: May 24, 2024
(86) PCT No.: PCT/IB2024/055080

§ 371 Date: Nov. 21, 2025

(30) Foreign Application Priority Data
May 26, 2023 (IT) 102023000010749

(51) Int. Cl. A61K 38/17; A61K 9/00; A61K 31/728; A61K 47/32; A61K 47/38; A61P 1/04

(52) U.S. Cl. CPC A61K 38/1767; A61K 9/0053; A61K 31/728; A61K 47/32; A61K 47/38; A61P 1/04

(57) ABSTRACT

The present invention relates to some new combinations of water-soluble hydrolyzed fibroin and hyaluronic acid, or a salt thereof, optionally together with a bioadhesive agent, the oral compositions including such combinations, and their use in therapy, in particular in the prevention and/or treatment of esophagitis and, in general, of the epithelial oesophagus damages.

The present invention relates to some new combinations of water-soluble hydrolyzed fibroin and hyaluronic acid, or a salt thereof, optionally together with a bioadhesive agent, the oral compositions including such combinations, and their use in therapy, in particular in the prevention and/or treatment of esophagitis and, in general, of the epithelial oesophagus damages.

### TECHNICAL BACKGROUND

Fibroin is an insoluble protein present in the silk produced by some insects, like Bombyx mori larvae, and other moths such as Antheraea, Cricula, Samia and Gonometa. Silk in its original form is constituted by two main proteins, sericin and fibroin, sericin being the sticky interleave between two filaments of fibroin. (Hakimi O et al “Spider and mulberry silkworm silks as compatible biomaterials”, Composites Part B: Engineering. 38 (3): 324-37, April 2007).

The water-soluble hydrolyzed fibroin derived from silk fibroin contains about 30% of free amino acids and oligopeptides. This hydrolyzed has a high ability to acquire water thanks to the characteristic aminoacidic composition, and when applied to the skin, forms a protective layer which gives softness and a distinct smoothness sensation; this is the main reason why the water-soluble hydrolyzed fibroin is used in cosmetics.

Hyaluronic acid is a natural linear polysaccharide belonging to the glycosaminoglycan family, constituted by the disaccharide repetition formed by glucuronic acid linked to N-acetylglucosamine. Hyaluronic acid is ubiquitarian in the living organisms and in the last decades has taken more attention in the pharmaceutical and cosmetic areas as component of various topical and oral formulations

### STATE OF THE ART

US 2005182022 (U.S. Pat. No. 6,924,273) discloses chondroprotective/relieving compositions, especially to be administered as animal feed inside the thoroughbred horses races and comprises hyaluronic acid alone or in the combination with chondroitin sulphate and/or glucosamine, together with a jelly agent like for example a cellulose derivative. In particular the document discloses a composition which comprises 1% by weight of sodium hyaluronate, 4% by weight of chondroitin sulphate and 1% by weight of carboxymethylcellulose

EP2296670 discloses the combination of chondroitin sulphate and hyaluronic acid for the preparation of medicaments against esophagitis. The patent claims a combination of 150-500 mg of chondroitin sulfate and 80-120 mg of hyaluronic acid and from 300 to 750 mg of a bio-adhesive agent, with a dynamic viscosity from 39 to 100 mPas·s

### OBJECTS OF THE INVENTION

It is an object of the present invention to provide for new combinations of active ingredients for oral use to be used in therapy.

It is another object of the invention to provide for pharmaceutical oral compositions comprising the combinations of the invention, together with one or more pharma or food grade carriers and/or excipients.

It is finally another object of the invention to provide for the use of the oral combinations and of the compositions of the invention in particular, but not limited to, in the prevention and/or treatment of esophagitis and, more in general of the oesophageal epithelium damages.

### BRIEF DESCRIPTION OF THE DRAWING

FIG. 1 shows the % AD of the composition of the invention and placebo, in presence of mucin film at pH 3 (A) and pH 7 (B), according to Example 8.

FIG. 2 shows the physical properties of a composition of the invention (A), a composition comprising only sodium hyaluronate but not water-soluble hydrolyzed fibroin (B), and a composition comprising sodium hyaluronate and chondroitin sulfate in lieu of water-soluble hydrolyzed fibroin (C), according to Example 9.

### SUMMARY OF THE INVENTION

The applicant has observed that fibroin in its hydrolyzed water-soluble form, especially in the presence of hyaluronic acid or one of its salts, shows a high efficacy in the protection of the oesophagus mucosa.

In particular, it was found that combinations of water-soluble hydrolyzed fibroin and hyaluronic acid or one of its pharmaceutically acceptable salts, preferably together with a pharmaceutically acceptable or food grade bioadhesive component, prevent and/or heal the esophagitis onset, for example in case of oesophageal reflux, and allow for the treatment of oesophagitis or of the pathologies where the damage of the oesophageal epithelium of different origins occurs, like for example produced by side effects of drugs.

It was further observed that such combinations show particular efficacy when formulated as liquid oral compositions having a dynamic viscosity from 200 to 5,000 mPa·s measured at 20° C. and at atmospheric pressure.

### DESCRIPTION OF THE INVENTION

According to one of its aspects, a subject-matter of the invention is a combination for oral use which consists in a water-soluble hydrolyzed fibroin and hyaluronic acid or one of its pharmaceutically acceptable or food grade salt.

“Water-soluble hydrolyzed fibroin” here is used to indicate a hydrolyzed water-soluble protein rich of free amino acids and low molecular weight peptides that is obtained from silk fibroin. Water-soluble hydrolyzed fibroins (herein also named “fibroin according to the invention” or only “fibroin”) are commercially available. A suitable water-soluble hydrolyzed fibroin according to the invention is of food grade, for example the one marketed by Shaanxi Yuantai Biological Technology Co., Ltd (China) under the name Hydrolyzed Silk Fibroin.

In the present invention, any type of amino acid or oligopeptide mixture derived from silk is suitable, provided it is water-soluble, i.e. that can be fully solubilized in water without leaving a solid residue.

Preferably, the hyaluronic acid is in salified form, more preferably in its sodium or potassium salified form, even more preferably in its sodium salified form.

Preferably, the hyaluronic acid is in salt form, more preferably in its sodium or potassium salt form and has a weight average molecular weight Mw from 0.1 to 2 MDa, more preferably from 0.1 to 1.3 MDa.

Hyaluronic acid and its salts are also commercially available.

According to the invention, the weight ratio of water-soluble hydrolyzed fibroin and hyaluronic acid or one of its salts as defined above is from 10/1 to 1/1 (w/w), preferably from 6/1 to 2/1 (w/w), more preferably from 5/1 to 4/1 (w/w), for example from 3/1 to 3.5/1 (w/w). A preferred ratio is about 3.3/1 (w/w).

According to another of its aspects, a subject-matter of the invention is a combination for oral use constituted by the water-soluble hydrolyzed fibroin and hyaluronic acid or one of its pharmaceutically acceptable salts or food acceptable grade salts, as defined above, together with one or more pharmaceutically acceptable bioadhesive compounds.

The bioadhesive compounds are known to the art for their ability to make the active ingredients associated thereto to adhere on the action sites for the local administration (topical or oral) of drugs or to cover portions of the body.

Illustrative examples of bioadhesive compounds are pharmaceutically acceptable polymers, preferably selected from: the poloxamers, like the copolymers of ethylene and propylene oxide, in particular the products known under the name of Lutrol®; polymers of polyvinylpyrrolidone type, like the polyvinylpyrrolidone Kw24-32, that corresponds to a molecular weight of about 40,000 Da; and cellulose derivatives, like for example the hydroxypropyl cellulose and hydroxyethyl cellulose.

Mixtures of bioadhesive compounds can be also utilized, for example mixtures of cellulose derivatives and of the type of polyvinylpyrrolidone.

According to another of its aspects, a subject-matter of the invention are oral pharmaceutical compositions comprising the combinations of the invention, together to one or more pharmaceutically acceptable carriers and/or excipients.

Even if it not expressly indicated, the combinations and the compositions of the invention are for oral use.

According to one embodiment, the compositions of the invention comprise the combinations of the water-soluble hydrolyzed fibroin and hyaluronic acid or one of its pharmaceutically acceptable or food grade salts, together with one or more pharmaceutically acceptable carrier and/or excipients.

According to one embodiment, the compositions of the invention comprise the combination of the water-soluble hydrolyzed fibroin and hyaluronic acid or one of its pharmaceutically or food grade acceptable salts, and one or more pharmaceutically acceptable bioadhesive compounds, together with pharmaceutically acceptable carriers and/or excipients.

The compositions of the invention are suitable for the oral administration and can be in tablets form, chewable tablets, gummy oral form, hard capsules, soft capsules, powders, granules, or in liquid form, like solutions or suspensions.

According to a preferred embodiment, the compositions of the invention are in water based, liquid form, and have a dynamic viscosity from 200 to 5,000 mPa·s, measured at 20° C. and at atmospheric pressure.

According to some embodiments, the liquid oral compositions of the invention have a dynamic viscosity from 200 to 5,000 mPa·s, measured at 20° C. and at atmospheric pressure, for example a dynamic viscosity selected among the following: from 200 to 4,000, from 200 to 1,000, from 200 to 2,000, from 500 to 2,000 or from 2,000 to 5,000. The dynamic viscosities here reported have been measured as described in the Experimental Section below.

The compositions of the present invention can be in form of single dosage unity or in multi-dose form, preferably in single dosage unity form.

Each dosage unity can, for example, contain from 100 to 1,000 mg of water-soluble hydrolyzed fibroin and from 50 to 200 mg of hyaluronic acid, preferably in one of its pharmaceutically acceptable or food grade salt form, more preferably the sodium salt.

According to a preferred embodiment, each dosage unit preferably contains from 200 to 600 mg of water-soluble hydrolyzed fibroin and from 60 to 150 mg of hyaluronic acid, preferably in one of its pharmaceutically acceptable or food grade salt form, more preferably the sodium salt.

Representative examples of dosage units can contain:
200 mg of water-soluble hydrolyzed fibroin and 150 mg of sodium hyaluronate;
250 mg of water-soluble hydrolyzed fibroin and 80 mg of sodium hyaluronate;
350 mg of water-soluble hydrolyzed fibroin and 50 mg of sodium hyaluronate;
250 mg of water-soluble hydrolyzed fibroin and 100 mg of sodium hyaluronate;
400 mg of water-soluble hydrolyzed fibroin and 75 mg of sodium hyaluronate;
   where such hyaluronic acid is preferably is in its pharmaceutically acceptable or food grade salt form, more preferably sodium salt.

According to one embodiment, the compositions of the invention, in addition to the water-soluble hydrolyzed fibroin according to the invention and to the hyaluronic acid, in the form of pharmaceutically or food grade acceptable salts, more preferably sodium salt, also comprise one or more bioadhesive compounds as defined above, in amounts from 1 to 3,000 mg, more preferably from 100 to 1,000 mg.

The skilled in the art can understand that the amounts of bioadhesive compounds depend on the target viscosity of the composition of the invention.

The skilled in the art of the pharmaceutical technology can easily select the carriers and the excipients to be included in the composition of the invention.

For illustrative purposes, the solid forms can include starches, cellulose and its derivatives; lubricants like talc, stearic acid, polyethylene glycol or magnesium stearate; diluents like talc, cellulose powder, lactose, corn starch or wheat starch, mannitol, sorbitol; disaggregating agents like microcrystalline cellulose or crospovidone; ligands like methylcellulose, sodium carboxymethylcellulose, alginic acid, alginates; sweeteners like saccharose, dextrose, mannitol, saccharine, xylitol, sorbitol: or aromas like synthetic or natural oils.

Carriers and excipients suitable for the chewable oral forms include for example lubricants, binders, sweeteners. aromas, bioadhesives and disintegrating agents.

Water is a carrier for the liquid compositions, but not the only one. Other carriers and excipients for liquid formulations, usually aqueous ones, suspensions or solutions, include for example antioxidants, like sodium metabisulfite or sodium sulfite, thickening agents like microcrystalline cellulose, hydroxypropyl cellulose, carboxymethylcellulose or polyvinylpyrrolidone.

The compositions of the invention can also contain preservatives like methyl parabens, ethyl parabens, sodium ethylendiaminotetracetic acid (EDTA), sodium benzoate or a basic salt of sorbic acid.

The compositions of the invention can also contain flavourings and/or sweeteners, like for example natural sugars or reduced sugars like saccharose, dextrose, xylitol, mannitol or sorbitol or synthetic products like sodium saccharin or aspartame; synthetic or natural oils, these latter extracted from plants, leaves, flowers, fruits and their combinations, like cinnamon, mint, anise and citrus fruits leaves, bitter almonds, citrus fruits like orange and/or lemon, oils from linden and grape fruit. The preferred flavourings are those giving a taste of mint or fruit, like grape fruit, cherry, or citrus fruits, in particular orange and lemon, aromas and their mixtures.

Also, chocolate, vanilla, or eucalyptus aromas and the fruit essence, in particular apple, pear, kiwi, peach, strawberry, cherry, apricot, orange, lemon and grape fruit, can be advantageously used.

All the excipients and the carriers used in the compositions of the invention are pharmaceutical or food grade, preferably pharmaceutical acceptable forms.

The preservatives, like sodium benzoate, ascorbic acid and its salts, in particular potassium sorbate, EDTA and its salts, can be present in the compositions at a concentration from 0.01 to 0.4% by weight, with respect to the total weight of the composition.

The synthetic sweeteners can be present in the compositions at a percentage from 0.1 to 5% by weight, with respect to the total weight of the composition, while the natural sweeteners, and optionally the reduced sugars can be present from 10 to 20%, preferably from 15 to 20% by weight, with respect to the total weight of the composition.

The flavouring can be supported in solid matrix and are in general present in an amount of 0.1-0.5% by weight, with respect to the total weight of the composition.

As already said, liquid compositions are preferred, water based, preferably having a dynamic viscosity from 200 to 5,000 mPa·s, at 20° C. at atmospheric pressure. Preferably the pH of such liquid compositions is from 4.8 to 5.2.

The viscosity of the liquid compositions of the invention is important because a product with a very low viscosity (for example 100 mPa·s or lower) could be easily washed out from the target site where the composition performs its action, the oesophagus. A too high viscosity will result in difficulties in the administration of the composition.

A preferred liquid composition is a water solution, that comprises:
a) water-soluble hydrolyzed fibroin at the concentration of from 2 to 6% by weight;
b) hyaluronic acid, preferably in the form of one of its pharmaceutically or food acceptable salts, preferably as sodium salt, at the concentration of from 0.6 to 1.5% by weight;
c) at least one bioadhesive component as defined above, at the concentration of from 0.01 to 2% by weight;
d) water;
e) optionally other pharmaceutically acceptable carriers and/or excipients.

The compositions disclosed above may have a dynamic viscosity from 200 to 5,000 mPa·s, at 20° C. at atmospheric pressure, for example a viscosity selected among the following: from 200 to 4,000, from 200 to 1,000, from 200 to 2,000, from 500 to 2,000 or from 2,000 to 5,000.

The liquid compositions of the invention can be packaged in multi-dosage unity forms, for example in sealed sachet stick-pack type, or in small plastic or glass bottles, or any other container provided by the current technology, including the multi-dosage containers, preferably equipped with an appropriate device for the needed dosage administration.

All the compositions of the invention can be administered once or more than once a day. The daily dosage depends on the age, sex, and on the healthy state of the subject to be treated and on the severity of the disease or impairment.

In general, it can be considered a daily dosage from 200 to 1,000 mg of water-soluble hydrolyzed fibroin according to the invention, for example from 400 to 600 mg of hyaluronic acid, preferably in form of one of its pharmaceutically or food acceptable salts, more preferably as sodium salt, for example for 100 to 300 mg, such a daily dosage being able to be divided in two or more administrations, for example two daily administrations.

A preferred daily dosage includes the administration of 500 mg of water-soluble hydrolyzed fibroin according to the invention, and 150 mg of hyaluronic acid, preferably in one of its pharmaceutically or food acceptable salts form, more preferably as sodium salt, for example divided in two administrations. However other dosages can be considered.

Applicant observed that the fibroin according to the invention has the ability to adhere to the oesophagus epithelium forming layers over the mucosa. In particular, fibroin according to the invention adhere to the epithelial cell layers, including the cells of the oesophagus mucosa. Further, in an experimental session, the applicant tested the fibroin/hyaluronic composition according to the invention against a placebo product containing hyaluronic acid without fibroin, in the adhesion model of the inclined plan, which consists of a plexiglass support that was previously coated with a biological substrate and can be inclined at different angles, in a close chamber to maintain the set temperature. Underneath, an electronic balance capable of registering weight at pre-set time points is placed. The test was performed as described in Example 8 below, and resulted in an increased adhesion of the fibroin/hyaluronic composition according to the present invention, compared to placebo solution.

According to another aspect, a subject-matter of the invention is a water-soluble hydrolyzed fibroin in the prevention and/or treatment of the damages of the oesophagus epithelium and for the prevention and/or treatment of oesophagitis of various origins, for example from gastroesophageal reflux, or derived from drugs side effects or inappropriate feeding.

According to another aspect, a subject-matter of the invention is a pharmaceutical composition comprising a water-soluble hydrolyzed fibroin, and its use in the prevention and/or treatment of the damages of the oesophageal epithelium and for the prevention and/or treatment of oesophagitis of various origin, for example from gastroesophageal reflux or derived from drugs side effects or from an inappropriate feeding.

Further, it was found by the Applicant that the combinations of the water-soluble hydrolyzed fibroin with hyaluronic acid increases the power of adhesion and induces also an anti-inflammatory activity.

The addition of a bioadhesive compound to the combination of water-soluble hydrolyzed fibroin and hyaluronic acid or one of its pharmaceutically or food acceptable salts, further increases the power of adhesion of the combination, providing increasing therapeutic results.

According to another aspect, a subject-matter of the invention is the use of the combinations and of the compositions of the invention in therapy, preferably the use in the prevention and/or the treatment of the damages of the oesophagus epithelium and in the prevention and/or treatment of oesophagitis of different origins, for example from gastroesophageal reflux or derived from drugs side effects or inappropriate feeding.

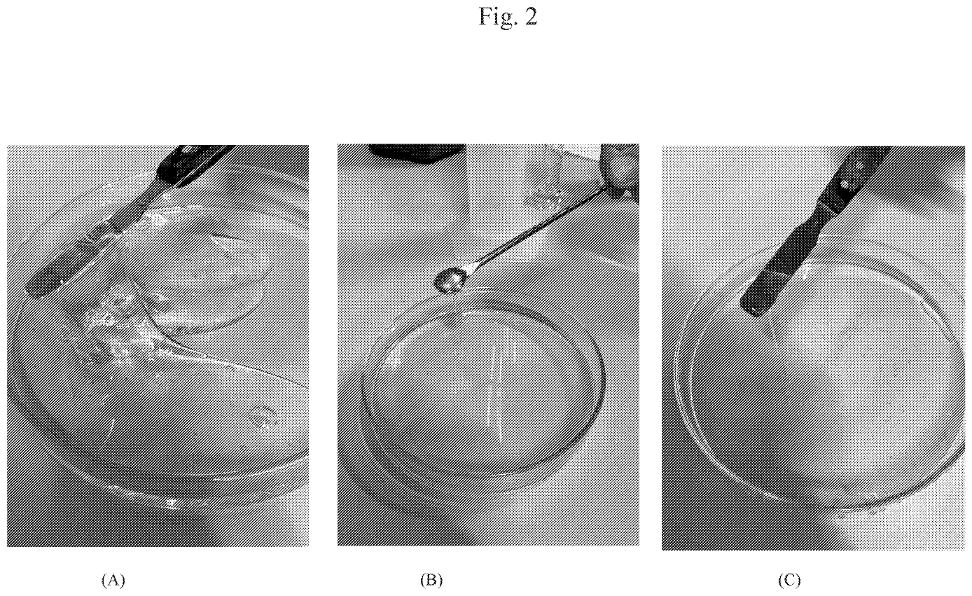

Applicant carried out experimental assays to compare the behaviour of a representative composition of the invention with respect to a composition containing only sodium hyaluronate and composition containing sodium hyaluronate and condroitin sulfate (in lieu of water-soluble hydrolyzed fibroin) at acidic pH, i.e. at a pH of the stomach and of the oesophagus in the presence of gastroesophageal reflux. The details of the experimentations are given in Example 9 below and the results are shown in FIG. 2. As it can be seen, the composition of the invention at acidic pH forms a cohesive gel, while no gel is formed with the composition comprising only sodium hyaluronate and a just a weak increase in viscosity was shown by the composition containing sodium hyaluronate and condroitin sulfate. The formation of a cohesive gel showed by the composition of the invention is completely unexpected and particularly advantageous, as it helps the composition to adhere to the oesophagus and stomach epithelium.

According to another aspect, a subject-matter of the invention is the method for the prevention and/or the treatment of the damages of the oesophagus epithelium and in the prevention and/or the treatment of oesophagitis of different origins, for example from gastroesophageal reflux or from drugs side effects or inappropriate feeding, which comprises to administer an efficacious dose of a combination of the invention to a subject in need thereof.

The combinations and compositions of the invention can be administered to mammals, preferably, but not only to humans, but also, for instance, to cats and dogs.

The composition of the invention can be prepared by a simple mixing of the active principles of the combinations and optionally of the carriers and excipients.

According to a preferred embodiment, the water base liquid compositions are prepared according to a process that comprises the dissolution of the bioadhesive compounds in water, optionally at reduced pressure and under stirring at a temperature from 40 to 80° C. and keeping under stirring for some time, for example 0.5-3 hours; the decrease of the temperature to 25-50° C. and the addition of the fibroin of the invention and the hyaluronic acid or one of its pharmaceutically or food acceptable salts; the decrease of the temperature to 25° C. and optionally the addition of the preservative, colouring agents and fragrances, previously dissolved in water; the correction of the pH of the solution so obtained to 4.5-5.5, preferably 4.8-5.2.

Some examples of preparation of the compositions of the invention are provided in the Experimental Section here below.

The procedure for the preparation of the water-based compositions of the invention is another subject-matter of the invention.

### EXPERIMENTAL SECTION

In the following examples:
The dynamic viscosity has been measured at 20° C., at atmospheric pressure by a rotational viscosimeter Brookfield DVII with spindle TF and at 15 RPM
The viscosity indicated for the hydroxyethyl cellulose was measured in water solution at the concentration of 2% at 20° C.

### Example 1

### Preparation of a Composition in Water-Based Liquid Form.

(i) Some sorbitol or xylitol are dissolved in water at 60° C. under strong stirring until a clear solution is obtained, if required a vacuum is applied up to 0.7 bar for 15 minutes. The solution is kept under mild stirring for 30 minutes. In the solution so obtained the bioadhesive polymer or the mixture of bioadhesive polymers are added always keeping a moderate stirring under vacuum up to 0.7 bar for 15 minutes. The stirring is kept for other 30 minutes, then the temperature is decreases to 40° C. and the water-soluble hydrolyzed fibroin and the sodium hyaluronate are added stepwise within 10-20 minutes. A yellowish clear solution is obtained and the temperature is decreased to 25° C.
(ii) Optional preservatives, colouring agents and the fragrances are dissolved in water under stirring at room temperature until a full dissolution;
(iii) To the solution obtained in step (i) the solution obtained in step (ii) is added and the mixture is kept under mild stirring at 25° C. for 15 minutes. The pH is measured and 4 M HCl is added to bring the pH in a range from 4.8 to 5.2

### Example 2

### Preparation of a Composition in Water Based Liquid Form.

(i) 1,800 gr of sorbitol and 6 kg of pure water are added in a mixer with paddle stirrer. The temperature is kept to 60° C. and the solution is vigorously stirred until a full dissolution. Then 100 gr of hydroxyethyl cellulose with low viscosity (100-400 mPa·s of 2% water solution), 100 gr of polyvinylpyrrolidone Kw 24-32 are added keeping the stirring and the vacuum for 15 minutes until a clear solution has obtained. The stirring is maintained for other 30 minutes, then the temperature is decreased to 40° C.

To this solution 250 gr of water-soluble hydrolyzed fibroin and 75 gr of sodium hyaluronate Shiseido batch SO55DA (weight average molecular weight=1 MDa) are added. The stirring is maintained until a yellowish clear solution has obtained. Then the temperature is decreased to 25° C.
(ii) In the meantime, 15 gr of sodium benzoate powder, 18 gr of potassium sorbate powder, 30 gr of aroma and 1612 gr of pure water are added in a mixer with magnetic stirring. A mild stirring is maintained until a clear solution has obtained.
(iii) The solution obtained in step (ii) is added to the mixer with the solution obtained in step (i), the mild stirring and the temperature at 25° C. are maintained for 15 minutes. The pH of the solution is 6.8, therefore the pH is reduced to below 5.2 with 4 M hydrochloric acid. 10 Kg of a solution to be filled in 1,000 units of dosage with the following dosage unit composition are obtained:

| | | |
|---|---|---|
| Water-soluble hydrolyzed fibroin | 250 | mg |
| Sodium hyaluronate (1 MDa) | 75 | mg |
| hydroxyethyl cellulose (100-400 mPa/s) | 100 | mg |
| Polyvinylpyrrolidone (Kw 24-32) | 100 | mg |
| Sorbitol | 1,800 | mg |
| Sodium Benzoate | 15 | mg |
| Potassium Sorbate | 18 | mg |
| Aroma | 30 | mg |
| Pure water to | 10,000 | mg |
| pH 5.04 | | |
| Dynamic viscosity 2,000 mPa/s | | |

### Example 3

Operating as described in example 2, but adding in the step (i) 75 gr of sodium hyaluronate with weight average molecular weight 0.5 Md, about 10 kg of solution to fill 1,000 dosage units are obtained with the following dosage unit composition:

| | | |
|---|---|---|
| Water-soluble hydrolyzed fibroin | 250 | mg |
| Sodium hyaluronate (0.5 MDa) | 75 | mg |
| hydroxyethyl cellulose (100-400 mPa/s) | 100 | mg |
| Polyvinylpyrrolidone (Kw 24-32) | 100 | mg |
| Sorbitol | 1,800 | mg |
| Sodium Benzoate | 15 | mg |
| Potassium Sorbate | 18 | mg |
| Aroma | 30 | mg |
| Pure water to | 10,000 | mg |
| pH 4.8 | | |
| Dynamic viscosity 326 mPa/s | | |

### Example 4

Operating as described in example 2, but adding in the step (i) 75 gr of sodium hyaluronate with weight average molecular weight 0.1 Md, about 10 kg of solution to fill 1,000 dosage units are obtained with the following dosage unit composition:

| | | |
|---|---|---|
| Water-soluble hydrolyzed fibroin | 250 | mg |
| Sodium hyaluronate (0.1 MDa) | 75 | mg |
| hydroxyethyl cellulose (100-400 mPa/s) | 100 | mg |
| Polyvinylpyrrolidone (Kw 24-32) | 100 | mg |
| Sorbitol | 1,800 | mg |
| Sodium Benzoate | 15 | mg |
| Potassium Sorbate | 18 | mg |
| Aroma | 30 | mg |
| Pure water to | 10,000 | mg |
| pH 5.4 | | |
| Dynamic viscosity 200 mPa/s | | |

### Example 5

Operating as described in example 2, but adding in step (i) 80 gr of sodium hyaluronate (weight average molecular weight=1 Md) 5 gr of hydroxyethyl cellulose (H30K 1,000-3,000 mPa·s) and 100 gr of polyvinylpyrrolidone (Kw 24-32), about 10 kg of solution to fill 1,000 dosage units are obtained with the following dosage unit composition:

| | | |
|---|---|---|
| Water-soluble hydrolyzed fibroin | 250 | mg |
| Sodium hyaluronate (1 MDa) | 80 | mg |
| hydroxyethyl cellulose (1,000-3,000 mPa/s) | 5 | mg |
| Polyvinylpyrrolidone (Kw 24-32) | 100 | mg |
| Sorbitol | 1,800 | mg |
| Sodium Benzoate | 15 | mg |
| Potassium Sorbate | 18 | mg |
| Aroma | 30 | mg |
| Pure water to | 10,000 | mg |
| pH 5.4 | | |
| Dynamic viscosity 1,500 mPa/s | | |

### Example 6

Operating as described in Example 2, adding in step (i) 80 gr of sodium hyaluronate (weight average molecular weight=1 Md) 10 gr of hydroxyethyl cellulose (H30K 1000-3000 mPa·s) and 100 gr of polyvinylpyrrolidone (Kw 24-32), about 10 kg of solution to fill 1,000 dosage units are obtained with the following composition:

| | | |
|---|---|---|
| Water-soluble hydrolyzed fibroin | 250 | mg |
| Sodium hyaluronate (1 MDa) | 80 | mg |
| hydroxyethyl cellulose (1,000-3,000 mPa/s) | 10 | mg |
| Polyvinylpyrrolidone (Kw 24-32) | 100 | mg |
| Sorbitol | 1,800 | mg |
| Sodium Benzoate | 15 | mg |
| Potassium Sorbate | 18 | mg |
| Aroma | 30 | mg |
| Pure water. to | 10,000 | mg |
| pH 5.4 | | |
| Dynamic viscosity 4,800 mPa/s | | |

### Example 7

In a pilot study, 18 patients without Helicobacter infection (10 females and 8 males) with ages from 18 to 80 years where selected. All patients showed oesophagitis and gastritis symptoms characterised by stomach burns, epigastric pain, dyspepsia, meteorism and with belching. Among those patients 11 had historical reflux problems inadequately treated with pump inhibitors and antiacids. The patients, to whom all previous treatments were suspended 5 days before the starting of the trial, have been treated with the composition of example 2. The composition has been administered with the following protocol: one 10 gr stick of solution twice a day for 4 weeks. The relief of symptoms has been defined as the reduction of pain or of the retrosternal burning, the reduction of the epigastric pain or burning, the reduction of the regurgitation or the sensation of acid in the mouth. All the patients filled a questionnaire at start and at the end of the trial and the data have been evaluated. The results obtained as statistically valid with p<0.05. The treatment was fully safe, nobody retired during the trial and the product compliance was very favourable. The effectiveness was very satisfactory. All patients have shown intensity and frequency reduction after 4 weeks of treatment, in particular 14 patients had an 85% reduction of all studied symptoms, 2 patients the 60% and 2 of 15%. The decrease of the acidic sensation in the mouth resulted statistically significative (p<0.05).

### Example 8

To evaluate the adhesion properties, the fibroin solution according to Example 6 was compared in the inclined plane test with a placebo solution prepared according to Example 6 but containing just sodium hyaluronate and no water-soluble hydrolyzed fibroin.

In this test method an inert surface is coated with a layer of mucus. Each test item is then applied on top and the plane is inclined to a certain angle, usually between 15 and 45 degrees. This mimics the contact of the test item and the mucus in vivo. The adhesion strength of the test item to the mucus prevents the solution from sliding off due to the force of gravity.

A gastric mucin suspension was used as a biological substrate. Mucoadhesion is a function of pH, ionic strength, and mucin concentration, therefore, given the intended use of the medical device, mucoadhesive properties were evaluated both at pH 7 and pH 3. This analysis was performed at a controlled temperature, specifically at 37° C. which is the expected temperature of the site of application of the test item.

### Preparation of Buffer Solutions:

Citrate and phosphate buffer solutions, characterized by pH values equal to 3 and 7 respectively, were prepared according to the European Pharmacopoeia. For pH 3 citrate buffer, sodium citrate dihydrate and citric acid were dissolved in MilliQ water, while, for pH 7 phosphate buffer, monobasic potassium phosphate and sodium hydroxide were solubilized in MilliQ water.

### Preparation of Mucin Suspensions:

Two different mucin suspensions were prepared by dispersing porcine gastric mucin in citrate buffer solution (MUC pH 3) or phosphate buffer solution (MUC pH 7) under magnetic stirring at room temperature. The final mucin concentration was 8% w/w.

### Inclined Plane Method

The inclined plane apparatus consists of a plexiglass support coated with a biological substrate and can be inclined at different angles in a close chamber to maintain the set temperature. Underneath an electronic balance capable of registering weight at pre-set time points is placed. to maintain a constant pre-set temperature.

The biological substrate consisted of a mucin film, which was prepared by casting 2.5 ml of a 8% w/w mucin suspension in pH 3 citrate buffer and pH 7 buffer, at 45° C. for 45 minutes. The test items Example 6 and placebo were analysed as such, and all the measurements were carried out at 37° C. An amount of each sample equal to 2 mL was placed onto the mucin film at the top of the support, still held in horizontal position; subsequently, the plane was inclined at 450 and the amount of formulation dropped on the microbalance was recorded after 3 minutes of time. Blank measurements were performed in absence of the biological substrate. Differential dropped percentage parameter (% ΔD) was considered as an index of sample mucoadhesive properties, and it was calculated according to the following equation:

% ΔD=% DROPPED without mucin film−% DROPPED with mucin film
wherein:
% DROPPED without mucin film=% amount of formulation dropped on the microbalance in absence of the biological substrate
% DROPPED with mucin film=% amount of formulation dropped on the microbalance in presence of artificial mucus.

Three replicates were considered for each sample.

### Results

TABLE
% dropped values for placebo and Example 6 with mucin

| | placebo-% dropped | Example 6-% dropped |
|---|---|---|
| with mucin pH 3 | 61.30 ± 0.95 | 51.77 ± 0.29 |
| with mucin pH 7 | 68.85 ± 0.19 | 62.78 ± 0.43 |
| without mucin | 81.36 ± 1.15 | 66.15 ± 1.52 |

In both cases, with mucin and without mucin, at pH 7 and especially at pH 3, the % dropped is lower for the solution according to Example 6.

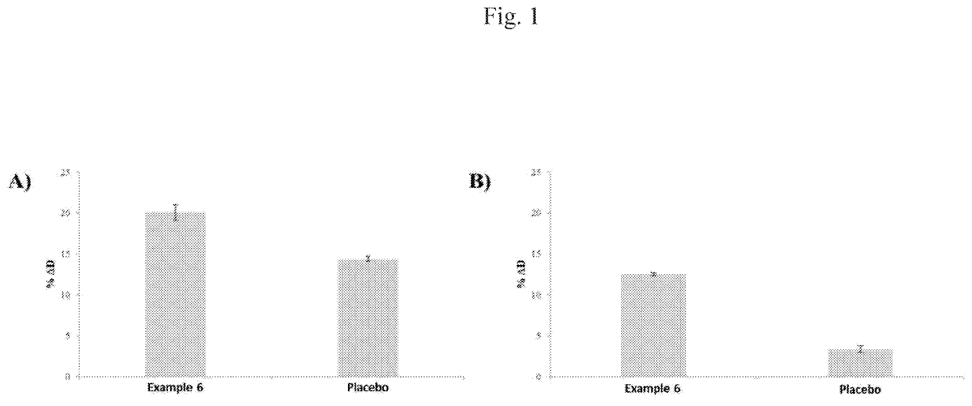

In FIG. 1, the mucoadhesive parameter % ΔD calculated for Example 6 and placebo, in presence of MUC pH 3 (Figure A) and MUC pH 7 (Figure B) are reported. % ΔD parameter higher than 0 in both the conditions considered indicates a mucoadhesive potential for the two samples. In both the experimental conditions when the normalization operated calculating the mucoadhesive parameter % ΔD, Example 6 showed a higher mucoadhesive potential than placebo. Both the batches were characterized by higher values in presence of MUC pH 3.

Inclined plane method measurements indicate that for both pH values considered, stronger interactions are established between the composition of the invention and mucins, even stronger at lower pH values, i.e. at pH similar to the one of the stomach and of the oesophagus in the presence of gastroesophageal reflux.

### Example 9 #

The fibroin composition according to Example 6 (Testing sample) was compared with a composition prepared according to Example 6 containing no water-soluble hydrolyzed fibroin (only sodium hyaluronate) (Reference 1), and with a composition prepared according to Example 6 containing chondroitin sulfate in place of water-soluble hydrolysed fibroin (Reference 2). The three samples had a final pH of 4,8-5,2 and a dynamic viscosity of 1000-2000 cP. The three solutions have been brought to pH 1 with 2 M hydrochloric acid. In these conditions the Testing sample (containing fibroin) formed a cohesive solid gel as reported in FIG. 2 (A), while Reference 1 did not show a gel formation at all (FIG. 2 (B)) and Reference 2 just showed an increase in viscosity (FIG. 2 (C)). Thus, the presence of water-soluble hydrolyzed fibroin, but not chondroitin sulfate, induces the formation of a cohesive gel at low pH.

1. A combination for oral use consisting of a water-soluble hydrolyzed fibroin, hyaluronic acid or a pharmaceutically acceptable salt thereof, and optionally one or more pharmaceutically acceptable bioadhesive compounds.

2. The combination according to claim 1, characterized in that said hyaluronic acid or a pharmaceutically acceptable salt thereof has a weight average molecular weight Mw ranging from 0.1 to 2 MDa, preferably from 100 to 1,300 KDa.

3. The combination according to claim 1 or 2, characterized in that the ratio, by weight, of the water-soluble hydrolyzed fibroin to the hyaluronic acid, or a pharmaceutically acceptable salt thereof, ranges from 10/1 to 1/1 (w/w), preferably from 6/1 to 2/1 (w/w), more preferably from 5/1 to 4/1 (w/w).

4. The combination according to any one of claims 1 to 3, characterized in that said one or more bioadhesive compounds are selected from: poloxamers, such as ethylene oxide and propylene oxide copolymers, preferably the products known under the trade name Lutrol®; polyvinylpyrrolidone type polymers, preferably Polyvinylpyrrolidone Kw24-32; and cellulose derivatives, preferably hydroxypropyl cellulose and hydroxyethyl cellulose.

5. A pharmaceutical composition for oral use comprising a combination according to any one of claims 1 to 4, together with one or more pharmaceutically acceptable carriers and/or excipients.

6. The composition according to claim 5, characterized in that it is in liquid form and has a viscosity ranging from 200 to 5,000 mPa·s measured at 20° C. and 25 atmospheric pressure.

7. The composition according to claim 5 or 6, characterized in that it is in unit dosage form and comprises from 100 to 1,000 mg water-soluble hydrolyzed fibroin and from 50 to 200 mg hyaluronic acid, preferably in form of a pharmaceutically acceptable salt thereof, more preferably from 200 to 600 mg water-soluble hydrolyzed fibroin and from 60 to 150 mg hyaluronic acid, preferably in form of a pharmaceutically acceptable salt thereof, more preferably sodium salt.

8. The composition according to any one of claims 5 to 7, characterized in that it is in unit dosage form and comprises 250 mg water-soluble hydrolyzed fibroin and 80 mg sodium hyaluronate, preferably in form of a pharmaceutically acceptable salt thereof, more preferably sodium salt.

9. The composition according to any one of claims 5 to 8, characterized in that it is an aqueous solution having a viscosity from 200 to 5,000 mPa·s, at 20° C., at atmospheric pressure, comprising:
a) water-soluble hydrolyzed fibroin at a concentration of 2 to 6% by weight;
b) hyaluronic acid, preferably in form of one of its pharmaceutically acceptable salts, more preferably in form of its sodium salt, at a concentration of 0.6 to 1.5% by weight;
c) at least one bioadhesive compound as defined above, at a concentration of 0.01 to 2% by weight;
d) water;
e) optionally further pharmaceutically acceptable carriers and/or excipients;
said percentages being expressed by weight with respect to the total weight of the composition.

10. The combination according to any one of claims 1 to 4 or composition according to any one of claims 5 to 9, for use in therapy, preferably in the prevention and treatment of damage to the epithelium of the oesophagus and in the prevention and/or treatment of esophagitis of different origins.

11. A water-soluble hydrolyzed fibroin for use in the prevention and/or treatment of damage to the epithelium of the oesophagus and in the prevention and/or treatment of esophagitis of different origins.
